(19) United States
(12) Patent Application Publication
(10) Pub. No.: US 2008/0311169 A1
(43) Pub. Date: Dec. 18, 2008

(54) Composition Comprising Ginsenosides for Treating or Preventing Angiostenosis and Restenosis

(75) Inventors: Sung-Sick Woo, Seoul (KR); Ji-Min Cha, Seoul (KR); Dong-Seon Kim, Daejeon (KR); Sun-Young Sung, Gyeonggi-do (KR); Seon-Gil Do, Chungcheongbuk-do (KR); Young-Chul Lee, Daejeon (KR); Kang-Woo Lee, Chungcheongnam-do (KR); Il-Hyoung Jung, Busan (KR); Soo-Kyung Sung, Chungcheongbuk-do (KR)

Correspondence Address: FITZPATRICK CELLA HARPER & SCINTO, 30 ROCKEFELLER PLAZA, NEW YORK, NY, 10112, US

(73) Assignee: UNIGEN INC., Cheonan-si (KR)

(21) Appl. No.: 11/629,086

(22) PCT Filed: Jun. 10, 2005
(86) PCT No.: PCT/KR05/01763

§ 371 Date: Dec. 11, 2006

(30) Foreign Application Priority Data
Jun. 11, 2004 (KR) 10-2004-0043008

(51) Int. Cl. A61K 9/00; A61K 31/704; A61K 36/254

(52) U.S. Cl. 424/423; 514/26; 424/728

(57) ABSTRACT

The present invention relates to use of ginsenoside Rg3, Rg5 or Rk1, or extract of ginseng, red ginseng or processed ginseng comprising the ginsenosides; a composition for preventing or treating angiostenosis and restenosis comprising the ginsenosides or extracts; a method for preventing or treating angiostenosis and restenosis by administrating the ginsenosides or extracts comprising the ginsenosides; and a preparation method of agents for preventing or treating angiostenosis and restenosis. The present composition can effectively prevent or treat angiostenosis and restenosis.

### TECHNICAL FIELD

The present invention relates to use of ginsenoside Rg3, Rg5 or Rk1 which is component of saponin system of ginseng having below structure, or extract of ginseng, red ginseng, or processed ginseng comprising the ginsenosides, a composition comprising the ginsenosides or extracts, a method for preventing or treating angiostenosis and restenosis by administrating the ginsenosides or extracts comprising the ginsenoside, and a preparation method of agents for preventing or treating angiostenosis and restenosis by extracting ginseng, red ginseng, or processed ginseng.

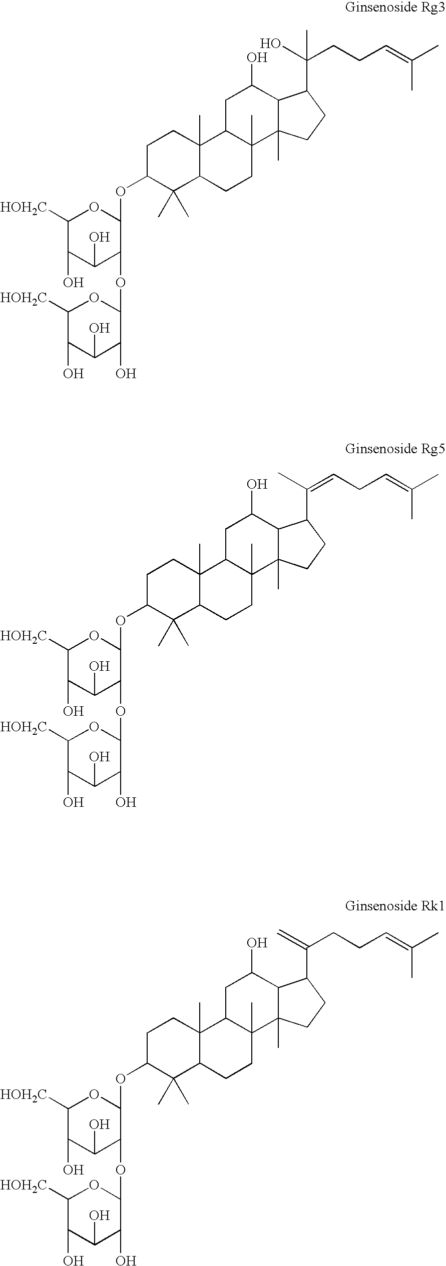

### BACKGROUND ART

Vascular disorder occurs in blood vessel, and blocks blood supplied to cardiac muscle. The most common cause of the vascular disorder is arteriosclerosis. Cholesterol and other fat, acute thrombosis, augmented plaque by combining other components in blood, and leucocyte activation and adhesion cause arterial stenosis, thereby reducing the supply of blood to cause shortage of oxygen [Libby P et al., Circulation, 86(6), 47-52, 1992, Lundgren C H et al., Circulation, 90(4), 1927-1934, 1994., Harker et al., Ann. NY Acad. Sci., 275, 321-329, 1976.]. As a result, stenocardia and cardiac infarction may be occurred, and in severe cases, result in death.

Now, the treatment for vascular disorder may be divided into treatment for angiogenesis and prevention for angiostenosis and restenosis by inhibiting the growth of muscle cell.

Percutaneous Transluminal Coronary Angioplasty is a method to dilate narrowed coronary artery without surgical operation, and consists of Percutaneous Coronary Balloon Dilation, Percutaneous Coronary Stent Insertion, etc. The Percutaneous Coronary Balloon Dilation is a method to improve the blood flow of the coronary artery in the following manner: a guide conduit is inserted through the femoral region and the artery of arms, and placed on the entrance of the coronary artery which has lesion through the aorta; another conduit on which a balloon is attached to is placed on the region of stenosis of the coronary artery through the guide conduit after confirming location of the guide conduit; the balloon is made to be dilated; and such dilated balloon has the narrowed coronary artery dilated by compressing plaque, etc., thereby improving the blood flow of the coronary artery. Also, the Stent Insertion method is to cover the inner wall of the coronary artery with wire netting by dilating wire netted balloon after such balloon is placed on the region of stenosis. The rate of restenosis of the Stent Method is lower than that of Balloon Angioplasty that only dilates the balloon. Stent is characterized in playing a support role to the inner wall of blood vessel, and so the Stent Method is used for the treatment of complications occurring in dilating the balloon.

This Coronary Angioplasty is used worldwide since it is more convenient than the surgery, can lower the risk of anesthesia, and has higher rate of success. This method can be preferably used for patients who have high risk in surgery or anesthesia due to old age, cardiac disease, and respiratory disease. In case of terminal cancer patient to gastrointestinal tract or biliary tract who cannot undergo surgical operation, this method can improve the quality of life by improving its body condition for the remaining period of life.

However, in case of the Coronary Angioplasty, restenosis is occurred due to injury of endothelium of blood vessel, mural thrombosis, movement of smooth muscle cell, fibroblast of blood vessel, permeation of mononuclear cell and lymphocyte, proliferation of cell in neointima, reendothelialization, apoptosis, etc., as a vegetation process of inner membrane of blood vessel induced by injury. In 6.8% of patients, restenosis may be occurred due to thrombosis or convulsion of blood vessel, and more severe restenosis of blood vessel may be occurred in 3 or 6 months after dilation surgery of blood vessel. Also, in 40% of patients, restenosis may be occurred around the operation area of balloon dilation (Herrman J-PR et al., Drugs, vol. 46, 18-52, 1993). In 20% of the blood vessel operated around artery and vein, the coronary artery, and the part of endarterectomy of femoral artery, the blood vessel can be blocked by secondary change of blood vessel [Volteas N et al., Int. Angiol, 2; 13(2), 143-147, 1997]. Also, restenosis may be frequently occurred in case of diabetes, old age, early stage of angina pectoris, or unstable angina pectoris (Leinigruber P P et al., Circulation, vol. 73, 710, 1986).

To prevent restenosis of coronary artery, new PTCA (Percutaneous Transluminal Coronary Angioplasty) equipments such as atherectomy, laser angioplasty, rotablator, cutting balloon angioplasty, and irradiation have been introduced. Also, various treatment methods such as systemic and local drug therapy of antiplatelet drug, antithrombotic, vasodilator, inhibitor of cell growth, agent for improving lipid metabolism, antioxidant, etc.; and molecular biology like genetherapy have been developed and tried. Among these methods, the systemic drug therapy such as oral administration or intravenous administration is most conveniently used, but is reported to be effective for the prevention of restenosis only in animal test. That is, the method could not prevent restenosis in clinical trial due to side effects of drugs, and desired level of drug could not be reached at the operation area of PTCA. Theoretically, restenosis is occurred only at the coronary artery of the operation area on which PTCA is performed, and so to prevent restenosis, the local drug therapy which can site-specifically administer highly concentrated drug is more useful than the systemic drug therapy.

Recently, for direct administration of drug to the operation area of PTCA, double balloon catheter, dispatch, microporous balloon, etc. are developed and used in clinic. Also, to deliver drug into the operation area of PTCA for a long period of time, slow release microsphere or treating with drug-coated stent has been tried more and more.

As known compositions for the treatment and prevention of restenosis, Korean Patent Publication No. 2001-84811 discloses catechin, extract of green tea, and Korean Patent Publication No. 2004-8013 discloses clotrimazole. Also, coating agents used frequently at present are rapamycin, paclitaxel, silorimus, verapamil, etc.

### DISCLOSURE OF THE INVENTION

Conventional drugs were expensive and limited in use, and there has been a need for superior medicines for the prevention and treatment of angiostenosis and restenosis that are cheap, can be easily applied to stent due to high solubility in organic solvent, and have activities of vasodilation as well as effects for inhibiting cell growth. Thus, the present inventors have repeated extensive studies to develop new agents for the prevention and treatment of angiostenosis and restenosis, and discovered that ginsenoside Rg3, Rg5, or Rk1, and the extract of red ginseng can be used for the prevention or treatment of angiostenosis and restenosis by inhibiting the growth of muscle cell. Based on this discovery, the present inventors confirmed that the extract of processed ginseng enriched with ginsenosides by specially processing ginseng and red ginseng is more effective for the prevention or treatment of angiostenosis and restenosis, to complete the present invention.

An object of the present invention is to provide a composition for the prevention or treatment of angiostenosis and restenosis comprising ginsenoside Rg3, Rg5 or Rk1, or the extract of ginseng, red ginseng, or processed ginseng containing these ginsenosides.

Another object of the present invention is to provide a method of prevention or treatment of angiostenosis and restenosis comprising adrninistering a therapeutically effective amount of ginsenoside Rg3, Rg5 or Rk1, or the extract of ginseng, red ginseng, or processed ginseng containing ginsenoside Rg3, Rg5 or Rk1 to the patients who need the prevention or treatment of angiostenosis and restenosis.

Another object of the present invention is to provide a use of ginsenoside Rg3, Rg5, or Rk1, or the extract of ginseng, red ginseng, or processed ginseng containing ginsenoside Rg3, Rg5 or Rk1 to prevent or treat angiostenosis and restenosis.

Another object of the present invention is to provide a method for preparing agents for the prevention or treatment of angiostenosis and restenosis by extracting ginseng, red ginseng, or processed ginseng containing ginsenoside Rg3, Rg5, or Rk1 with water, C₁₋₄ alcohol, or mixing solvent thereof.

### BRIEF DESCRIPTION OF THE DRAWINGS

FIG. 1 is a graph showing the effect of cell toxicity due to ginsenoside Rg3, Rg5 or Rk1.

FIG. 2 is a graph showing the effect of cell toxicity due to the extract of red ginseng and the present composition.

FIG. 3 is a graph showing the effect of growth inhibition of muscle cell due to ginsenoside Rg3, Rg5, or Rk1.

FIG. 4 is a graph showing the effect of growth inhibition of muscle cell due to the extract of red ginseng and the present composition.

### BEST MODE FOR CARRYING OUT THE INVENTION

To accomplish the objects described above, the present invention provides a composition for the prevention or treatment of angiostenosis and restenosis comprising ginsenoside Rg3, Rg5, or Rk1 as an active ingredient.

The above composition may be prepared by using pure ginsenosides Rg3 and/or Rg5 and/or Rk1; or the extract of ginseng or red ginseng comprising these ginsenosides; or processed ginseng or extract thereof enriched with these ginsenosides.

The present invention also provides a ginseng composition for the prevention or treatment of angiostenosis and restenosis comprising the extract of ginseng, red ginseng, or processed ginseng containing ginsenoside Rg3, Rg5, or Rk1.

The above extract of ginseng or red ginseng is not particularly limited, but preferably is an extract of water or C₁₋₄ alcohol such as methanol, ethanol, propanol, butanol, etc., or mixing solvent thereof, and can be prepared by conventional methods from raw ginseng.

The present invention also provides health care products comprising the above composition.

The present invention also provides a stent coated with the above composition.

The present invention also provides a method of prevention or treatment of angiostenosis and restenosis comprising admninistering a therapeutically effective amount of ginsenoside Rg3, Rg5, or Rk1, or the extract of ginseng, red ginseng, or processed ginseng containing these ginsenosides to the patients who need prevention or treatment of angiostenosis and restenosis.

The present invention also provides a use of the extract of ginseng, red ginseng, or processed ginseng containing ginsenoside Rg3, Rg5, or Rk1 to prevent or treat angiostenosis and restenosis.

The present invention also provides a method for preparing agents for the prevention or treatment of angiostenosis and restenosis by extracting ginseng, red ginseng, or processed ginseng containing ginsenoside Rg3, Rg5, or Rk1 with water, C₁₋₄ alcohol, or mixing solvent thereof.

In the present invention, ginseng can be selected from the group comprising Panax ginseng, P. japonicum, P. quinquefolium, P. notoginseng, P. trifolium, and P. pseudoginseng, without limitation, and used by root, stem, leaf, or herb.

The ginseng extract enriched with the above ginsenoside Rg3, Rg5, or Rk1 can be obtained by the treatment of acid, enzyme, or high temperature from root, leaf, top, and flower of ginseng containing ginseng saponin; tissue culture material of ginseng; or extract thereof extracted with water or lower alcohol.

In one embodiment of the present invention, the above processed ginseng is obtained by the method that, i) ginseng is treated with acid at 50˜80° C., and ii) the treated ginseng is steamed at the temperature under 110° C. for 0.5˜15 hr.

For example, the composition of the present invention can comprise the extract of processed ginseng or lyophilized product thereof. The extract is prepared by extracting processed ginseng with water, or common organic solvent such as lower alcohol of C₁₄, wherein the processed ginseng is prepared by two steps of: i) treating ginseng with acid at 50˜80° C. (1^{st} step) and ii) steaming the treated ginseng of 1^{st} step at the temperature under 10° C. for 0.5˜15 hr (2^{nd} step).

The present composition can be prepared by additionally mixing the above extract of processed ginseng or lyophilized product thereof with powdered red ginseng or white ginseng, which is clearly within the scope of the present invention.

In the present invention, the acid which can be used in the 1^{st} step of the above processing method is not particularly limited so long as the acid can cause substitution of the substituent located at 20^{th} carbon of the ginsenoside of ginseng, but preferably, acetic acid. In case of using acetic acid, the concentration of acetic acid is not particularly limited, but may be 20˜100%. Particularly, it is preferable to use acetic acid because the boiling point of acetic acid is about 107° C., and so it can be removed in the steaming process without additional removal process.

In the acid treatment of the 1^{st} step, it is preferable for the steaming temperature to be about 50˜80° C., more preferably 65˜75° C., since the substitution by acid can be promoted in the temperature range. Also, it is preferable to treat ginseng at 70° C. with acid for 0.1˜10 hr, more preferable 1˜5 hr, particularly preferable 3 hr.

In the present invention, the processed ginseng is prepared by steaming the ginseng treated in 1^{st} step at the temperature under 110° C. for 0.5˜15 hr. In case of the processing method described in Korean Patent No. 96-17670, there is a practical drawback that the range of temperature should be maintained at 120˜180° C., which lowers economic efficiency. The present invention is much more convenient than the above patent method, and can increase the contents of ginsenosides Rg3, Rg5 and Rk1 with high yield because the ginseng in the present invention is steamed at the temperature under 110° C., preferably 100° C., more preferably 100˜20° C., for 0.5˜15 hr, preferably 0.5˜8 hr, more preferably 1˜3 hr.

In the present invention, the extract or lyophilized product thereof used for the present composition may be prepared by conventional methods such as broth extraction or sonication with using solvents like water, C₁₋₄ alcohol such as methanol, ethanol, propanol, butanol, etc., or mixed solvents thereof, after the above 2^{nd} step.

The composition of the present invention can be used for agents of prevention or treatment of angiostenosis and restenosis by remarkably improving angiostenosis and restenosis as shown in the experimental example below.

The composition of the present invention can be prepared according to conventional methods in the pharmaceutical field. That is, the present composition can be prepared into conventional preparations, for example, solution such as drinks, syrup, and capsule, mixed with pharmaceutically acceptable carrier, excipient, etc.; and administered orally or parenterally. Preferably, the present composition may be orally administered in drinks before and/or after the meal for prompt effect.

Preferably, capsule and solution comprising the present composition may be used as health care products. Here, “health care products” mean food products prepared and processed in the form of tablet, capsule, powder, granule, solution, pill, etc., by using material or ingredients having useful function to the human body.

The composition of the present invention may be appropriately selected according to the extent of absorption of active ingredients in the body; excretion rate; age, weight, sex, and condition of patient; severity of treated disease, etc., but generally, it is preferable to administer the present composition as solution 1˜3 times a day, 0.5˜10 ml/kg each. Other forms of preparations may be orally administered in an appropriate amount considering the above amount for solution.

Hereinafter, the present invention will be described in more detail with reference to the following examples, but the scope of the present invention should not be construed to be limited thereby in any manner.

### EXAMPLES

### A: Preparation of 20(R) & 20(S) Ginsenosides Rg3, Rg5, and Rk1

Chromatography was conducted to 30 g of powder of processed ginseng extract on silica gel column by using lower layer of methylenechloride/methanol/water (v/v, 75:30:10) as eluent, to obtain 600 mg of fraction containing ginsenoside Rg3 and 400 mg of fraction containing ginsenosides Rg5 and Rk1.

600 mg of fraction containing ginsenoside Rg3 was recrystallized with methanol, to obtain 150 mg of 20(R) ginsenoside Rg3. Ginsenoside Rg3 was isolated from 400 mg of the other methanol soluble fraction by using the Preparative HPLC system of HITACHI Co. (pump; L-7100, detector; L-7455, interface; D-7000, column oven; L-7300, automatic feeder; L-7200). The condition of isolation was as follows: Zorbax Eclipse XDB-C18 9.4*250 mm was used as stationary phase; the condition of mobile phase was acetonitrile/Water (v/v, 40:60); the flow rate was 4 nm i/min; the total time of isolation was 90 min; and the sample was dissolved in methanol at the concentration of 100 mg/ml, injected by 50 μl, and detected by UV detector at 203 nm. Preparative HPLC was repeatedly preformed to obtain 60 mg of ginsenoside (S)—Rg3.

20(R) Ginsenoside Rg3 ¹³C-NMR (ppm, pyridine-d₅): δ 15.73, 16.31, 16.51, 17.21, 17.63, 18.35, 22.52, 22.71, 25.77, 26.53, 26.64, 28.02, 31.31, 32.05, 35.07, 36.8, 39.01, 39.61, 39.90, 43.16, 49.10, 50.27, 50.49, 51.67, 56.25, 62.56, 62.73, 70.79, 71.49, 71.51, 72.89, 77.07, 77.84, 78.04, 78.21, 78.37, 83.34, 88.81, 105.05, 105.98, 125.95, 130.71

20(S) Ginsenoside Rg3 ¹³C-NMR (ppm, pyridine-d₅): δ 15.73, 16.26, 16.31, 16.45, 17.39, 18.32, 22.39, 25.24, 26.12, 26.24, 26.64, 27.51, 30.72, 31.43, 35.27, 36.29, 37.34, 38.51, 39.09, 39.97, 47.94, 49.76, 51.10, 54.19, 55.74, 62.05, 62.21, 70.40, 70.99, 72.36, 72.40, 76.55, 77.33, 77.52, 77.80, 78.21, 82.80, 88.30, 104.52, 105.45, 126.95, 130.16

400 mg of fraction containing ginsenosides Rg5 and Rk1 was isolated by using the Preparative HPLC system of HITACHI Co. (pump; L-7100, detector; L-7455, interface; D-7000, column oven; L-7300, automatic feeder; L-7200). The condition of isolation was as follows: Zorbax Eclipse XDB-C18 9.4*250 mm was used as stationary phase; the condition of mobile phase was acetonitrile/Water (v/v, 48:52); the flow rate was 4 ml/min; the total time of isolation was 90 min; and the sample was dissolved in methanol at the concentration of 100 mg/ml, injected by 50 μl, and detected by UV detector at 203 nm. Preparative HPLC was repeatedly preformed to obtain 30 mg of ginsenoside Rg5 and 10 mg of ginsenoside Rk1.

Ginsenoside Rg5 ¹³C-NMR (ppm, pyridine-d₅): δ 13.0, 16.0, 16.5, 16.6, 17.0, 17.8, 18.5, 25.8, 26.7, 27.0, 27.4, 28.1, 32.3, 32.6, 35.3, 37.0, 39.2, 39.7, 40.2, 50.5, 50.9, 51.2, 56.4, 62.6, 62.8, 71.4, 72.4, 72.6, 77.2, 77.9, 78.1, 78.3, 78.3, 83.5, 88.9, 105.2, 106.1, 123.5, 124.6, 131.2, 140.2

### B: Preparation of Processed Ginseng

1. Acetic Acid Treatment Process

The steaming instrument [Seogang ENG (Inc.), Korea], concentrator (YELA, Japan), lyophilizer [Ilshinwrap (Inc.), Korea] used for preparing processed ginseng was owned by the Material Development Team of UNIGEN, Inc. Raw ginseng of 4-years-roots (Keumsan) was used as raw material for processing. Also, to compare the contents of ginsenosides Rg3 and Rg5, besides the raw ginseng of 4-years-roots, red ginseng, white ginseng, white tail ginseng, and raw ginseng of 5-years-roots (Keumsan) were purchased and used. Anhydrous acetic acid of more than 95% [Samjeon Chemistry (Inc.), Korea] was used as a solvent for reaction of acetic acid.

To estimate optimum concentration for acetic acid treatment and optimum treating method, 100 g of raw ginseng of 4-years-roots was quantified, and put into each of two plastic containers, and 1.5 L of 50% of acetic acid mixed with anhydrous acetic acid and water at the rate of 1:1, and 1.5 L of 100% of anhydrous acetic acid not mixed with water were put into each of the plastic container. One plastic container among the two plastic containers containing acetic acid was heated in water-bath at 70° C. for 3 hr, and the other plastic container was left at the room temperature for 2, 4, 6, 8, 10, 24 and 48 hr without heating.

2. Steaming of ginseng and preparation of the extract

The processed ginseng prepared in the above step B.1. was steamed and extracted for removal of acetic acid, addition of sugar, and hydrolysis. To determine the optimal temperature and time of steaming, the raw ginseng treated with acetic acid was steamed under the temperature and time of 120° C. (8 hr), 100° C. (3 hr), 80° C. (8 hr), and 80° C. (3 hr), to result in Samples 9˜12.

The processed ginseng was extracted with 70% of ethanol at 80° C. for 6 hr, and then extracted at 45° C. for about 5 hr.

To reduce the pumping effect due to the rising of temperature and decompression during the lyophilization, the extracts, which are reactants with high viscosity (65-70 Brix), were diluted with warm water to lower the viscosity to about 24 Brix, and then the reactants were cooled at −70° C. for 2 days. When the cooling of the reactants was completed, the reactants were lyophilized at −70° C. and 10 mtorr for 2 days.

3. Contents analysis

The HITACIH system (pump; L-7100, detector; L-7455, interface; D-7000, column oven; L-7300, automatic feeder; L-7200) was used as HPLC for analysis of the reactants. The condition of analysis was as follows: Capcell PAK C18 (5 μm), 3.0*75 mm was used as stationary phase; the condition of mobile phase was gradient control with solvent A of acetonitril, and solvent B of Water; the flow rate was 0.5 ml/min; the total time of isolation was 110 min; the temperature of column oven was 40° C.; the dosage of sample was 101 l; and the sample was detected by UV detector at 203 nm. Ginsenosides Rb1, Rb2, Rc, Rd, Re, Rf and Rg1 were isolated within 60 min, and Rg3, Rg5 and Rk1 were isolated after 70 min.

The samples for analysis of the processed ginseng extract prepared in the above step B. 2. were prepared with methanol to the concentration of 2 mg/ml. The standard samples of ginsenosides were prepared in the concentration of 0.2 mg/ml. Both the standard samples and raw material samples treated with acetic acid were put into automatic feeder, and analyzed.

Raw ginseng of 4-years-roots was processed by using the above acetic acid treatment method according to the conditions in Table 1 (samples 18) below, and the results thereof were analyzed as follows: in case of no heating treatment, Rg3 and Rg5 were hardly formed, and in case the heating treatment was done at 70° C. for 3 hr, Rg3 and Rg5 were formed in the 50% of acetic acid reactant and 100% of acetic acid reactant, respectively. Here, 50% of acetic acid was proven to be the optimal concentration of acetic acid since it is advantageous to minimize the concentration of acetic acid as reaction solvent considering up-scale mass manufacturing process. Also, heating treatment was decided as the treating method.

TABLE 1
Change of ginsenoside content according to pretreatment
of acetic acid and treating method

| Sample | Rb1 | Rb2 | Rc | Rd | Re | Rf | Rg1 | (R)-Rg3 | (S)-Rg3 | Rg5 | Rk1 |
|---|---|---|---|---|---|---|---|---|---|---|---|
| 1 | 13.40 | 4.95 | 6.10 | 2.69 | 9.24 | 2.07 | 3.87 | 0.17 | — | — | — |
| 2 | 20.19 | 7.27 | 6.69 | 4.08 | 15.64 | 2.98 | 7.78 | — | — | — | — |
| 3 | 22.85 | 8.82 | 7.97 | 4.86 | 17.26 | 3.17 | 9.28 | — | — | — | — |
| 4 | 23.61 | 9.44 | 7.82 | 4.91 | 16.90 | 3.46 | 9.28 | — | — | — | — |
| 5 | 23.50 | 9.01 | 8.41 | 4.96 | 17.09 | 3.46 | 9.30 | — | — | — | — |
| 6 | 24.44 | 9.41 | 12.86 | 5.11 | 16.81 | 3.47 | 9.25 | — | — | — | — |
| 7 | 0.46 | — | — | — | — | — | — | 6.75 | 3.47 | 10.45 | 3.24 |
| 8 | 1.25 | — | — | — | — | — | — | 5.46 | 3.84 | 11.78 | 3.77 |

Sample 1: No retting in acetic acid;
Sample 2: Retting in 50% of acetic acid for 2 hr;
Sample 3: Retting in 50% of acetic acid for 4 hr;
Sample 4: Retting in 50% of acetic acid for 6 hr;
Sample 5: Retting in 50% of acetic acid for 8 hr;
Sample 6: Retting in 50% of acetic acid for 10 hr;
Sample 7: Heating in 50% of acetic acid at 70° C., for 3 hr;
Sample 8: Heating in 100% of acetic acid at 70° C., for 3 hr

Comparing the contents of ginsenosides according to the steaming process, relatively high contents of ginsenosides Rg3 and Rg5 were shown at the condition of 120° C. for 8 hr and 100° C. for 3 hr. And, the acetic acid was completely removed at 100° C. In terms of economic efficiency, the temperature and time should be minimum. Therefore, the steaming temperature of 100° C. and the steaming time of 3 hr were assessed to be optimal in the process (Table 2).

TABLE 2
Change of ginsenoside content according to the steaming process

| Sample | Rb1 | Rb2 | Rc | Rd | Re | Rf | Rg1 | (R)-Rg3 | (S)-Rg3 | Rg5 | Rk1 |
|---|---|---|---|---|---|---|---|---|---|---|---|
| 9 | 0.35 | — | — | — | — | 0.72 | — | 15.39 | 13.43 | 29.26 | 12.38 |
| 10 | 0.43 | — | 0.53 | — | — | 0.04 | — | 17.35 | 15.37 | 31.33 | 11.89 |
| 11 | 0.32 | — | 0.42 | — | — | 0.01 | 0.10 | 7.33 | 6.21 | 8.42 | 3.15 |
| 12 | 0.18 | — | 0.40 | — | 0.12 | 0.07 | 0.05 | 9.17 | 7.62 | 9.68 | 4.73 |

Sample 9: Treating with 50% of acetic acid (heating at 70° C. for 3 hr), and then steaming at 120° C. for 8 hr;
Sample 10: Treating with 50% of acetic acid (heating at 70° C. for 3 hr), and then steaming at 100° C. for 3 hr;
Sample 10: Treating with 50% of acetic acid (heating at 70° C. for 3 hr), and then steaming at 80° C. for 3 hr;
Sample 10: Treating with 50% of acetic acid (heating at 70° C. for 3 hr), and then steaming at 80° C. for 8 hr

The contents of ginsenosides according to the kind of ginseng showed similar for all red ginseng, white ginseng, and raw ginseng (Table 3).

TABLE 3
Change of ginsenoside content according to the kind of ginseng (mg/g)

| Sample | Rb1 | Rb2 | Rc | Rd | Re | Rf | Rg1 | (R)-Rg3 | (S)-Rg3 | Rg5 | Rk1 |
|---|---|---|---|---|---|---|---|---|---|---|---|
| 13 | 8.14 | 25.38 | 25.31 | 19.28 | 5.74 | 5.59 | 11.21 | 1.58 | 0.64 | 1.09 | 0.35 |
| 14 | 0.24 | — | — | — | — | — | — | 18.34 | 16.28 | 32.78 | 13.72 |
| 15 | 7.88 | 15.04 | 13.05 | 12.32 | 2.67 | 4.61 | 7.88 | 1.28 | 0.22 | 0.17 | 0.05 |
| 16 | 0.45 | — | — | — | — | — | — | 12.15 | 10.28 | 21.83 | 7.14 |
| 17 | 8.63 | 10.83 | 5.36 | 1.94 | 2.19 | 0.39 | 0.95 | 1.60 | 0.05 | 0.05 | 0.02 |
| 18 | 0.35 | — | — | — | — | — | — | 13.39 | 11.74 | 24.05 | 9.36 |
| 19 | 36.42 | 34.91 | 26.59 | 9.84 | 14.57 | 2.38 | 5.26 | 6.86 | — | 1.55 | 0.48 |
| 20 | 0.69 | — | — | — | — | — | — | 18.15 | 15.28 | 29.78 | 11.24 |

Sample 13: Red ginseng;
Sample 14: Treating red ginseng with 50% of acetic acid (heating at 70° C. for 3 hr), and then steaming at 100° C. for 3 hr;
Sample 15: White ginseng;
Sample 16: Treating white ginseng with 50% of acetic acid (heating at 70° C. for 3 hr), and then steaming at 100° C. for 3 hr;
Sample 17: White tail ginseng;
Sample 18: Treating white tail ginseng with 50% of acetic acid (heating at 70° C. for 3 hr), and then steaming at 100° C. for 3 hr;
Sample 19: Raw ginseng of 5-years-roots;
Sample 20: Treating raw ginseng of 5-years-roots with 50% of acetic acid (heating at 70° C. for 3 hr), and then steaming at 100° C. for 3 hr

In short, the method for preparing particular ginsenoside by using enzyme among the prior patent and preparation methods to increase the contents of ginsenosides Rg3 and Rg5 is not easily applicable to mass manufacturing process because the unit production cost is high, and the manufacturing process steps are complicated. Also, in the method inducing hydrolysis of ginsenosides under high temperature and pressure, it is difficult to establish the condition of high temperature and pressure.

In that sense, the present invention has an advantage in preparing the present composition to contain high content of ginsenoside by using solvent such as acetic acid without establishing the condition of high temperature and pressure. Also, the manufacturing process of the present composition is simpler than others, and thus it is easily applicable to up-scale mass manufacturing process. And, in the recovery and disposal of acetic acid, the process of the present invention is not using and separating a large amount of acetic acid, but is heating the raw material soaked in a certain amount of 50% of acetic acid. Also, used acetic acid is not disposed and can be reused in the state, and so the present invention complies with the environmental requirement under the MSDS.

### EXPERIMENTAL EXAMPLE

### 1. Cell Culture

SMCs (human aortic smooth muscle cells, Cambrex, USA) were cultured in SmGM-2 BulletKit (Cambex, USA) medium containing 10% FBS (Cambrex, USA), with 100× antibiotics (Cambex, USA) added thereto, and subcultured by using 1×trypsin-EDTA (Gibco BRL, USA) with maintaining the condition of 37° C., 5% CO₂.

### 2. Experiment of Cell Cytotoxicity

Before confirming whether ginsenoside and red ginseng extract affect the growth of muscle cell, the cell cytotoxicity was determined as the effect of cell cytotoxicity induced by ginsenosides Rk1, Rg3, and Rg5, red ginseng extract, and the present composition in muscle cell.

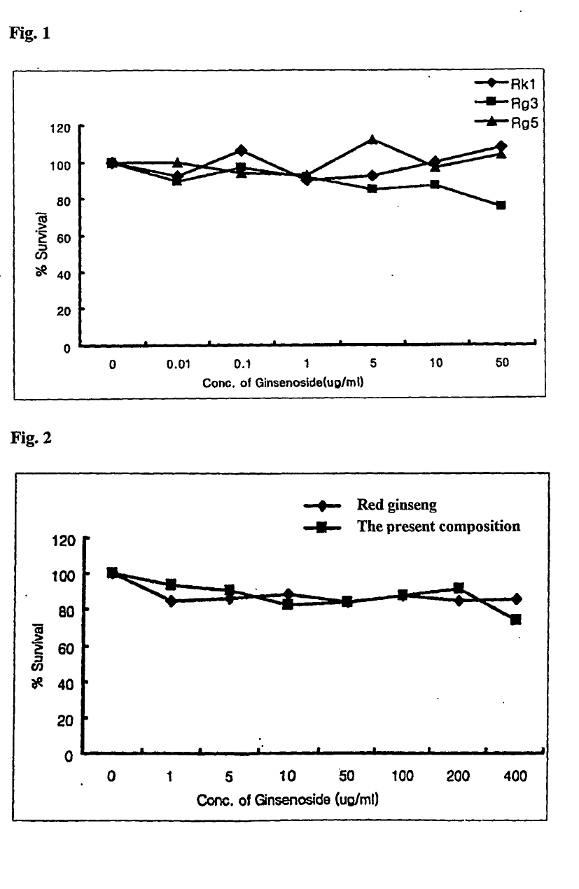

The cell cytotoxicity to the sample was determined by colorimetric MTT assay (Scudiero D. A. et al., Cancer Res., 48:4827-4833, 1988). That is, the muscle cell was plated to 96 wells microtiter tissue culture plate (Falcon) by 1×10⁴ cells/ml, and then each well was treated with the sample, cultured for a certain period of time, treated with MTT sample, and melted with solubilization solution when formazan was formed, and the absorbance was determined at 540 nm. The results were shown in the following FIGS. 1 and 2.

As shown in FIGS. 1 and 2, no cell cytotoxicity was found in proper concentration of ginsenosides Rk1, Rg3, and Rg5, red ginseng extract, and the present composition.

### 3. Experiment of Cell Proliferation

In the present experiment, the inhibition effects of cell proliferation by ginsenosides, red ginseng extract, and the present composition in muscle cell were determined.

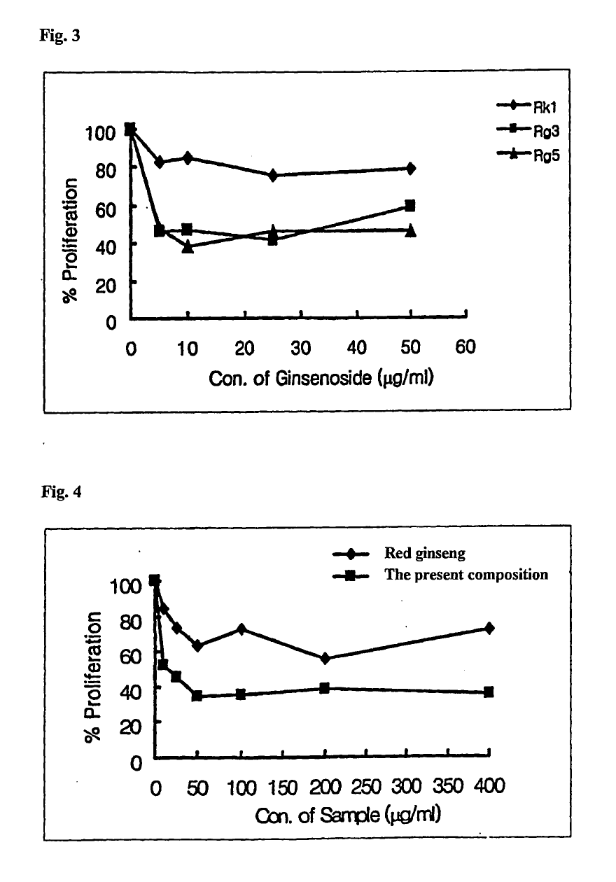

To determine the effects of cell proliferation by the sample, Cell proliferation ELISA BrdU assay kit (Roche, USA) was purchased and used. Cells were put into 96 wells plate, treated with the sample after cultivation, and cultured for a certain period of time. At a certain point of time, BrdU labeling solution was added into the cells, which in turn were reacted at 37° C., in 5% CO₂ for 2 hr. Then, FixDenat was added thereto, and the mixture was reacted at the room temperature for 30 min. And, Anti-BrdU-POD working solution was added thereto, and the mixture was reacted at the room temperature for 1 hr and 30 min. Afterwards, substrate solution was added thereto, and the mixture was reacted at the room temperature for 20 min. The reaction was stopped by using sulphuric acid, and then the absorbance was determined at 450 nm. The results were shown in the following FIGS. 3 and 4.

As shown in FIGS. 3 and 4, ginsenosides Rg3 and Rg5 inhibited 50% of the growth of muscle cell at the concentration of 5 μg/ml. Also, ginsenosides, red ginseng extract and the present composition inhibited the growth of muscle cell concentration-dependently. However, it was proven that the extract of processed ginseng containing more contents of ginsenosides Rg3, Rg5, and Rk1 inhibited the growth of muscle cell more powerfully than red ginseng extract (RG).

In sum, it was proven that ginsenosides Rg3, Rg5 and Rk1 inhibited the growth of muscle cell. Also, the extract of red ginseng and the extract containing ginsenosides inhibited the growth of muscle cell. Therefore, it can be said that these experimental samples are effective for the inhibition of neointima formation, and the prevention and treatment of angiostenosis and restenosis.

### FORMULATION EXAMPLE 1

### Preparation of Solution

| Ethanol Extract of Sample 14 | 20 | g |
|---|---|---|
| Sugar | 10 | g |
| Isomerized sugar | 10 | g |
| Smell of lemon | proper quantity | |
| Total amount after adding purified water | 100 | ml |

The above-mentioned ingredients were mixed according to conventional preparation method for solution, and sterilized to give solution.

### FORMULATION EXAMPLE 2

### Preparation of Solution

| Ethanol Extract of Sample 16 | 30 | g |
|---|---|---|
| Sugar | 10 | g |
| Isomerized sugar | 10 | g |
| Smell of lemon | proper quantity | |
| Total amount after adding purified water | 100 | ml |

The above-mentioned ingredients were mixed according to conventional preparation method for solution, and sterilized to give solution.

### FORMULATION EXAMPLE 3

### Preparation of Solution

| Ginsenoside Rg5 | 3 | g |
|---|---|---|
| Sugar | 10 | g |
| Isomerized sugar | 10 | g |
| Smell of lemon | proper quantity | |
| Total amount after adding purified water | 100 | ml |

The above-mentioned ingredients were mixed according to conventional preparation method for solution, and sterilized to give solution.

### FORMULATION EXAMPLE 4

### Preparation of Capsule

| | | |
|---|---|---|
| Ethanol Extract of Sample 14 | 500 | mg |
| Lactose | 50 | mg |
| Starch | 50 | mg |
| Talc | 2 | mg |

| | |
|---|---|
| Magnesium Stearate | proper quantity |

The above-mentioned ingredients were mixed, and filled in a gelatin capsule according to conventional preparation method for capsule to give capsule.

### FORMULATION EXAMPLE 5

### Preparation of Capsule

| | | |
|---|---|---|
| Ginsenoside Rg3 | 100 | mg |
| Lactose | 50 | mg |
| Starch | 50 | mg |
| Talc | 2 | mg |

| | |
|---|---|
| Magnesium Stearate | proper quantity |

The above-mentioned ingredients were mixed, and filled in a gelatin capsule according to conventional preparation method for capsule to give capsule.

### FORMULATION EXAMPLE 6

### Preparation of Drink

A mixture was prepared by mixing 6 weight % of lyophilized product of ethanol extract of Sample 20, 5 weight % of fructose, 0.1 weight % of citric acid, and a proper amount of lemon flavor, and purified water was added thereto to give drink.

### INDUSTRIAL APPLICABILITY

Ginsenoside Rg3, Rg5 or Rk1; or the extract of ginseng, red ginseng, or processed ginseng containing these ginsenosides are effective for the prevention or treatment of angiostenosis and restenosis. Particularly, processed ginseng containing more content of ginsenoside Rg3, Rg5 or Rk1 is much more effective for the prevention or treatment of angiostenosis and restenosis. The composition of the present invention can effectively prevent and treat heart diseases, etc. without surgical operation such as the Percutaneous Transluminal Coronary Angioplasty.

1. A composition for preventing or treating angiostenosis and restenosis comprising ginsenoside Rg3, Rg5 or Rk1 as effective ingredient.

2. A composition for preventing or treating angiostenosis and restenosis comprising the extract of ginseng, red ginseng, or processed ginseng containing ginsenoside Rg3, Rg5 or Rk1 with water, C₁₋₄ alcohol, or mixing solvent thereof.

3. A composition for preventing or treating angiostenosis and restenosis comprising the extract of water, C₁₋₄ alcohol, or mixing solvent thereof, of processed ginseng obtained by steaming treated ginseng at the temperature under 110° C. for 0.5˜15 hr, wherein the treated ginseng was obtained by treating ginseng with acid at 50˜80° C.

4. The composition according to claim 3, characterized in that the treated acid is acetic acid.

5. Health care products for preventing or treating angiostenosis and restenosis comprising the composition according to any of claims 1 to 4.

6. Health care products according to claim 5, selected from capsule, tablet, suspension, granule, solution, or powder.

7. Stent coated with the composition according to any of claims 1 to 4.

8. A method of preventing or treating angiostenosis and restenosis, comprising administering a therapeutically effective amount of ginsenoside Rg3, Rg5 or Rk1 to patients who need prevention or treatment of angiostenosis and restenosis.

9. A method of preventing or treating angiostenosis and restenosis, comprising administering a therapeutically effective amount of extract of water, C₁₋₄ alcohol, or mixing solvent thereof, of ginseng, red ginseng, or processed ginseng containing ginsenoside Rg3, Rg5 or Rk1, to patients who need prevention or treatment of angiostenosis and restenosis.

10. A method of preventing or treating angiostenosis and restenosis, comprising administering a therapeutically effective amount of extract of water, C₁₋₄ alcohol, or mixing solvent thereof, of processed ginseng obtained by steaming treated ginseng at the temperature under 110° C. for 0.5˜15 hr wherein the treated ginseng was obtained by treating ginseng with acid at 50˜80° C., to patients who need prevention or treatment of angiostenosis and restenosis.

11. The method according to claim 10, characterized in that the treated acid is acetic acid.

12. The method according to claim 10, characterized in that the processed ginseng contains ginsenoside Rg3, Rg5 or Rk1.

13. (canceled)

14. (canceled)

15. (canceled)

16. (canceled)

17. (canceled)

18. A method for preparing agents for preventing or treating angiostenosis and restenosis by extracting ginseng, red ginseng, or processed ginseng containing ginsenoside Rg3, Rg5 or Rk1 with water, C₁₋₄ alcohol or mixing solvent thereof.

19. The method according to claim 18, characterized in that the above processed ginseng is obtained by treating ginseng with acid at 50˜80° C., and steaming the treated ginseng at the temperature under 110° C. for 0.5˜15 hr.

20. The method according to claim 18, characterized in that the treated acid is acetic acid.
